(19) United States
(12) Patent Application Publication
(10) Pub. No.: US 2010/0150785 A1
(43) Pub. Date: Jun. 17, 2010

(54) SANITIZING DEVICE

(76) Inventors: Daniel Woolman, London (GB); Stuart Woolman, Charlotte, NC (US)

Correspondence Address: EDWARDS ANGELL PALMER & DODGE LLP, P.O. BOX 55874, BOSTON, MA, 02205, US

(21) Appl. No.: 12/598,019

(22) PCT Filed: Apr. 21, 2008
(86) PCT No.: PCT/GB08/01409

§ 371 Date: Oct. 28, 2009

(30) Foreign Application Priority Data
Apr. 30, 2007 (GB) 0708328.0
Oct. 15, 2007 (GB) 0720095.9
Oct. 23, 2007 (GB) 0720617.0

(51) Int. Cl. B65F 7/00; A61L 2/22

(52) U.S. Cl. 422/116; 422/292

(57) ABSTRACT

A sanitizing device (1) for a portable refuse container comprises a housing (3), an atomizing device (17) within the housing (3) for containing sanitizing material, a nozzle (11) attached to the housing (3) for dispersing the sanitizing material outside of the housing, means connecting the atomising device to the nozzle, timing means controlling the operation of the atomising device and means for releasably attaching the housing to the portable refuse container. The invention also includes a bracket suitable for releasably attaching a device, such as a sanitizing device, to the container.

### BACKGROUND OF THE INVENTION

This invention relates to a sanitizing device for a portable refuse container, and in particular to those commonly known as a “Wheelie Bin”.

There are hygiene problems associated with the use of wheelie bins. Rubbish is a breeding ground for disease. Bacteria can multiply extremely rapidly given the right conditions, Bacteria can grow and divide every 20 minutes. One single bacteria can become more than 8 million bacteria in less than 24 hours. Most of the germs that can be found in decaying food stuffs and other discarded organic matters can be found in wheelie bins. These include bacteria that can cause food poisoning such as Escherichia Coil, Listeria monocytogenes and Campylobacter jejuni.

Moreover the presence of vermin and flies in the bins represents a real danger to the end user and any children or immuno compromised persons living in close proximity. Flies are known carriers of germs and diseases including intestinal worms and gastroenteritis.

### BRIEF SUMMARY OF THE INVENTION

The present invention seeks to provide a sanitizing device which can be easily fitted to portable refuse containers, such as wheelie bins and which provides a sanitizing action on the bin contents. It also seeks to provide a bracket which has a universal application to attaching sanitizers to a wheelie bin.

According to a first aspect of the invention, a sanitizing device for a portable refuse container comprises a housing, an atomizing device within the housing for containing sanitizing material, a nozzle attached to the housing for dispersing the sanitizing material outside of the housing, means connecting the atomising device to the nozzle, timing means controlling the operation of the atomising device and means for releasably attaching the housing to the portable refuse container.

The nozzle may be positioned either on the housing or on the end of a flexible pipe connected to the atomising device whereby the nozzle can be arranged to be spaced from the housing.

Two or more nozzles, for example three, may be provided to give full cover to the contents of the portable refuse container.

The nozzles may be flexibly attached to the device by flexible pipes and more than three nozzles may be provided.

The device may have one or more hooks extending upwardly from the housing whereby the device can be hooked onto a lip of the portable refuse container so as to suspend the housing within the container.

The timer may be electric and may be settable to time predetermined intervals. The timer may be adjustable so as to vary the length of the timed intervals.

The atomising device, which may comprises an aerosol container containing the sanitizing material, may be electrically operated and may be triggered by the timer.

According to a second aspect of the invention, a bracket for attachment of a device to the upper part of a container comprises a back plate, attachment wings provided in mirror image on both sides of the back plate and intended to cooperate with the slots in the rear of the device such that, when the bracket is in position, the device can be engaged on the wings and pushed parallel to the bracket to seat the device on the bracket, a flat portion extending from the top of the back plate extending at right angles to the back plate by way of a “U” shaped connecting part and a front plate intended to extend down the outside of the container and means for securing the bracket to a wall of the container.

The back plate of the bracket may be provided with an aperture into which a detent on the device is intended to pass so as to secure the device to the bracket The bracket may have the dimensions stated in tables 1 and 2 and this may be plus or minus ten percent.

The bracket may be securable to the wall of the container by means of a screw which is threaded through the front plate suitably by way of a captive nut, and may be arranged so that its tip will engage the wall of the container so as to trap the container wall between the tip and the rear plate

### BRIEF DESCRIPTION OF THE DRAWINGS

The invention will now be described in greater detail, by way of example, with reference to the drawings, in which:—

FIG. 1 is a perspective view of one embodiment of the invention;

FIG. 2 is a perspective view of the housing of this embodiment taken from above with the top cover removed to show the contents of the housing;

FIG. 3 is a perspective view of the casing taken from the rear to show the attachment means of the housing;

FIG. 4 is a perspective view of a bracket suitable for use with the device;

FIG. 5 shows various views of the bracket showing the dimensions;

FIG. 6 is a detailed circuit diagram of the invention;

FIG. 7 is a simplified perspective view with a particular arrangement of the nozzle, and

FIG. 8 is a simplified perspective view with an arrangement of nozzles distributed and spaced from the device.

### DETAILED DESCRIPTION OF THE PREFERRED EMBODIMENTS

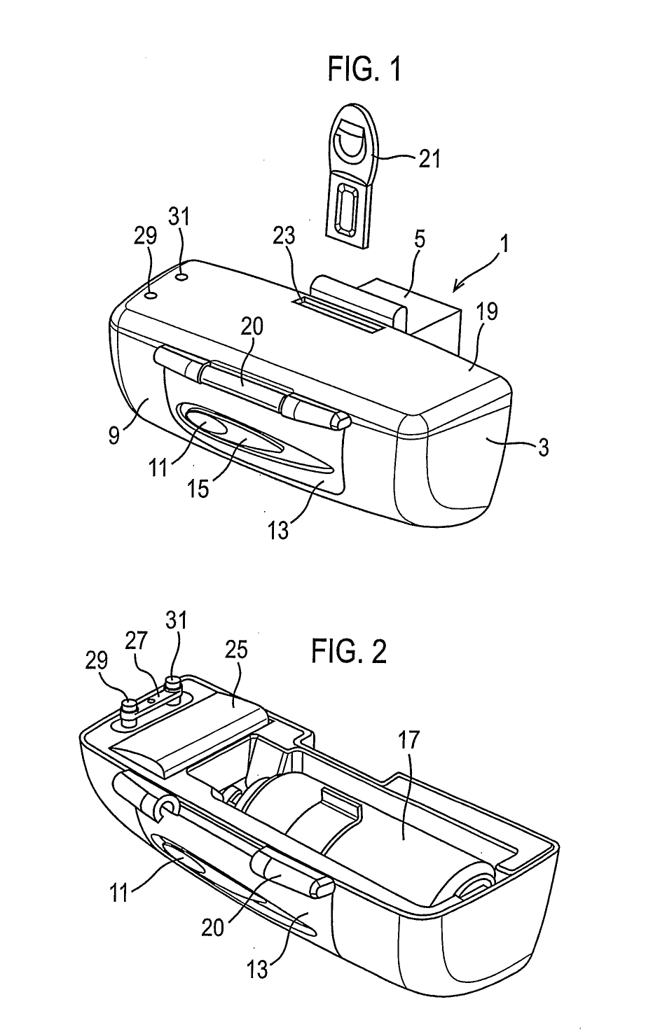

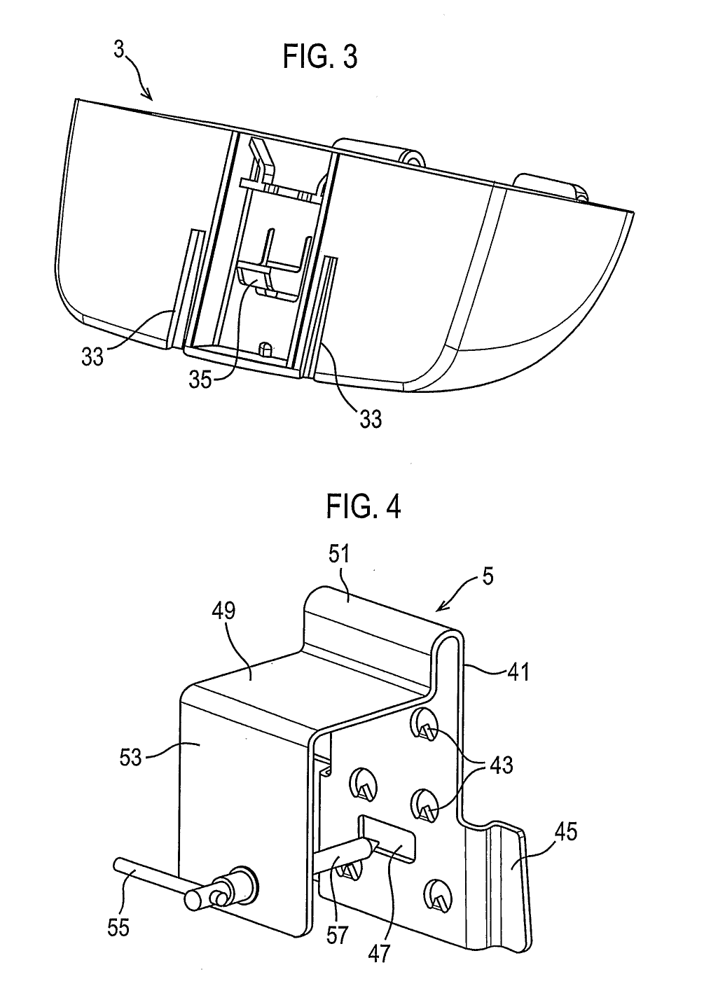

Referring to FIGS. 1 and 2 of the drawings a sanitizing device 1 for a portable refuse container such as a wheelie bin comprises an housing 3 which is adapted to be located on the inside of the container towards the top thereof. The housing 3 is made from a suitable water resistant plastics material. To support the housing 3, the housing has attached to it a bracket 5 which is intended to hook over the lip of the container and to be fastened there as will be described in more detail with respect to FIGS. 3, 4 and 5.

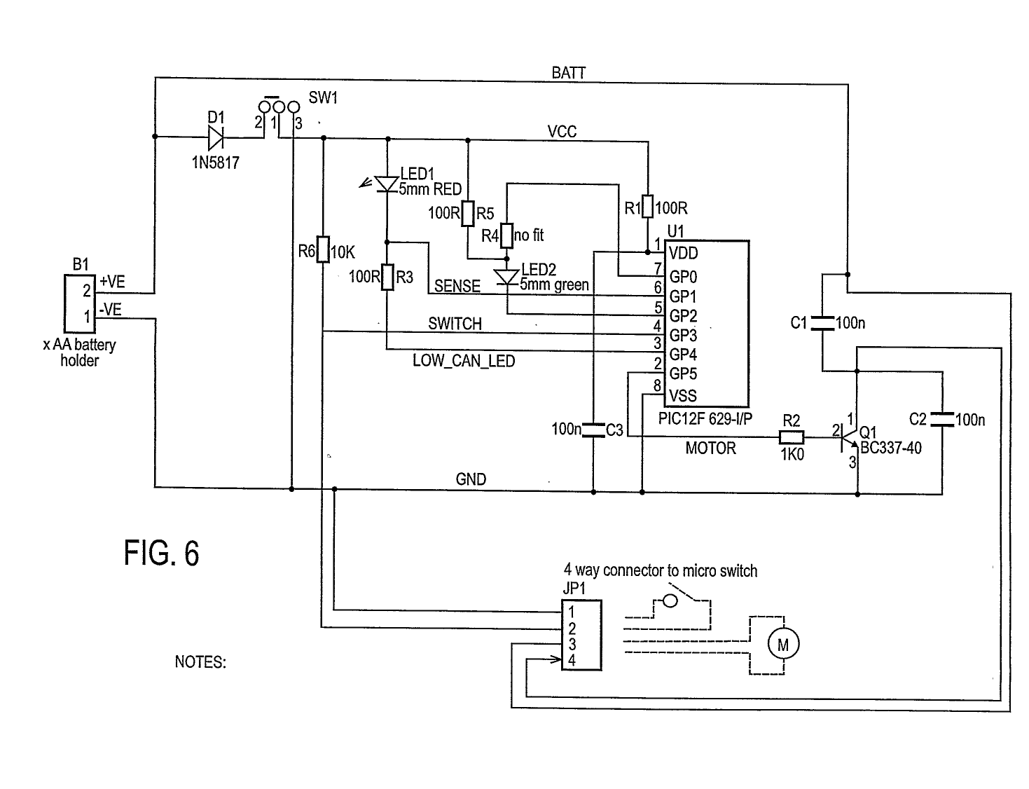

The front 9 of the housing 3 is provided with a nozzle 11 which is located behind a nozzle cover 13 and from which the sanitizing material is sprayed into the interior of the container. The cover 13 is provided with shaping 15 by means of which the spray from the nozzle 9 is directed over a wide area of the inside of the wheelie bin. The sanitizing material is carried by an aerosol container 17 which is connected to the nozzle by a line (not shown) so that, when the aerosol container 17 is activated, an atomised spray of sanitizing material is sprayed into the bin. The aerosol container 17 may be pressurised or non pressurised as desired. While one nozzle 11 is shown, a number of nozzles could be provided to assist in providing a better coverage of the spray. The nozzles could be carried by flexible pipes so that the nozzles could be located in any suitable position in the wheelie bin as is indicated in the embodiment of FIG. 6.

The housing 3 is covered by a hinged lid 19, hinged to the housing 3 by an hinge 20. The hinge 20 is so constructed that pressure downwards on it will cause the lid to be firmly engaged so that any rubbish hitting the lid 19 will not remove the lid 19. To this end, the hinge elements 28 are provided with downwardly facing undershot grooves into which the pivot pins (not shown) on the lid 19 are pushed upwardly in a click in fashion. The housing 3 can be locked in place on the bracket 5 and released by a key 21, insertable into a key slot 23. When the key 21 is inserted, housing 3 will be unlocked as will be explained hereafter. Next to the aerosol container is a compartment, seen closed by a lid 25, which compartment contains a motor and drive for actuating the aerosol, the electric circuitry (shown in FIG. 4) and an electrical battery. Also provided on the housing are an on-off switch 27 (beneath the lid 19), two LEDs 29 and 31 which extend upwards through the lid 19 and as will be described in relation to FIG. 6

At the rear of the housing 3, shown in FIG. 3 are the fixing arrangements for fixing the housing 3 to the bracket 5. These comprise two vertical slots 33 into which wings on the bracket 5 are slid and a sprung detent 35 which cooperates with an aperture in the bracket. The operation of these features will be described hereafter.

The bracket 5 is shown in FIG. 4. This bracket comprises a back plate 41 which has a number of barbs 43 which extend inwardly of the bracket for engaging the wall of the wheelie bin. Attachment wings are provided in mirror image on both sides of the bracket, only the right hand one of which is shown at 45. These wings 45 cooperate with the slots 33 in the rear of the housing so that, when the bracket 5 is in position, the housing can be engaged on the wings 45 and pushed downwards to seat the housing 3 on the bracket 5. The housing 3 is retained in position by the engagement of the detent 35 in an aperture 47. Unlocking of the housing 3 from the bracket 5 can be achieved by the use of the key 21. In use, this key is pressed down between the back plate 41 and the housing 3 whereby the detent 35 is pushed inwards, out of the aperture 47, releasing the housing so that it can be pushed upwards in respect of the bracket 5 and enable the housing to be removed. Extending from the top of the back plate 41 is a flat portion 49 extending at right angles to the back plate by way of a “U” shaped connecting part 51. This flat portion 49 is adapted to sit on the rim of the wheelie bin. Extending downwards from the flat portion 49 is a front plate 53 intended to extend down the outside of the wheelie bin and the bracket is secured to the wall of the wheelie bin by means of a screw 55 which is threaded through the front plate 53, for example, using a captive nut (not shown). and is arranged so that its tip 57 engages the wall of the wheelie bin so as to trap the wheelie bin wall between the tip 57 and the rear plate 41 to secure the bracket to the wheelie bin and thus securing the whole device thereto.

The bracket may be made out of any suitable material. In the form shown, it is pressed out of sheet steel but it could, with suitable modification, be made from a suitable plastics material. In the latter case, strengthening ribs would probably required. A two part construction could be employed with the front plate being separated from the rest of the bracket and being movable towards and away from the back plate in a similar fashion to a vice.

It will be understood that there are a very large number of different ways in which the device can be attached to the wheelie bin and any suitable means may be used. For example, the housing 3 could be secured to the wheelie bin wall by screwing or adhesives if there is no need to detach it therefrom. The positioning of the housing could be anywhere near the top of the wheelie bin or in positions down the sides, front, or back thereof. Additional possibilities include the use of more than one bracket with or without securing screws. A further possibility lies in providing wings on the housing which would extend right across the wheelie bin and be secured against the walls by pressure against the walls, for example by spring means or by actual attachment to the walls.

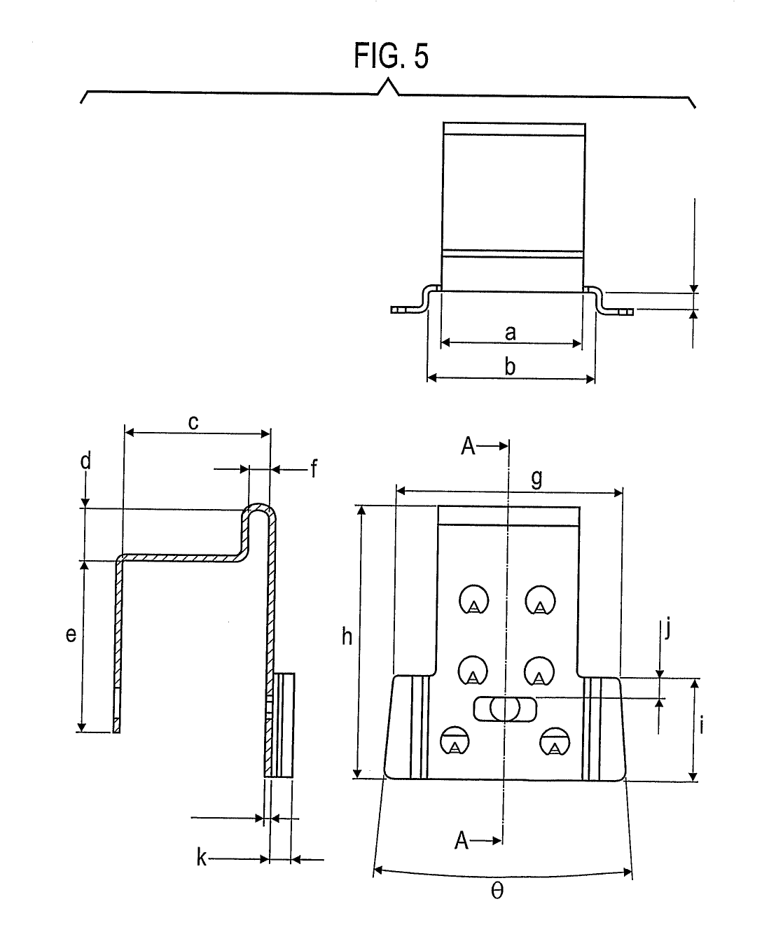

The dimensions of the bracket 5 are of some importance with regard to its universal application and FIG. 5 shows the bracket 5 with dimensions as indicated ion the following tables:—

TABLE 1

| Drawing reference | dimensions mm |
|---|---|
| a | 50 |
| b | 60 |
| c | 53 |
| d | 17 |
| e | 58.5 |
| f | 8 |
| g | 82 |
| h | 92 |
| i | 35 |
| j | 7.25 |
| k | 7.5 |

TABLE 2

| Drawing reference | Angle |
|---|---|
| θ | 10° |

It is to be understood that the invention includes measurements within 10% of the figures in the above tables.

The circuit of the device is shown in FIG. 6.

The function of the circuit is to drive a motor for short periods every few hours. The motor and associated gears and cam are used to press the release plunger of a metered aerosol canister, thus releasing a single dose of product from the device. The circuit also counts the number of doses and gives an indication when the canister is running low and empty (red LED). An ‘ON’ indication is shown when the batteries are in good condition (green LED). An off-circuit microswitch is used to sense the presence of a canister. This prevents the motor driving when there is no canister and is also used as a reset mechanism when the maximum dose count has been reached. A slide switch provides basic on/off function.

The printed circuit board (PCB) containing the circuit contains the electronics and battery holder for controlling the aerosol dispensing unit. The motor and canister-sensing microswitch are mounted off-board and connected via a 4-way plug/socket.

Power is derived from two AA (LR6) alkaline cells, using a PCB-mounted battery holder B1. This gives a nominal 3v supply. This power supply is fed directly to one side of the motor M to minimise power loss, and to the main circuit via schottky diode D1. This is to provide reverse-polarity protection in the event of someone placing batteries the wrong way round. Slide switch SW1 is the main on/off control. When the switch is ‘off’, the circuit supply is kept shorted to ensure that when the switch is moved to the ‘on’ position, the supply comes up from zero and produces a clean reset of the microcontroller chip U1.

All logic control and functionality is controlled by the microprocessor chip U1. This is a PIC12F629 8-bit device obtainable from Microchip. It is a miniature computer, with built-in software that has been written to control the sanitizing device. It has some memory that is maintained without power. This ‘non-volatile’ memory is used to keep count of the number of aerosol doses. This memory must be non-volatile as the unit may be switched off or batteries removed and the unit must still remember the dose count.

The supply to the microcontroller is filtered by resistor R1 & capacitor C3 to reduce the noise caused by the motor running. The microcontroller uses its built-in ‘watchdog’ circuit to reset the chip at regular intervals. Each time the chip resets, the software is executed, after which the chip ‘sleeps’ to conserve power until it is woken up by the next watchdog reset. This method is used to ensure minimum power consumption and longest battery life. Each reset is nominally 3 seconds apart. The interval will vary from chip to chip but this is compensated for by the software. Each reset interval is measured and therefore accurate timekeeping is maintained. The reset interval is effectively ‘calibrated’ automatically. Different chips will all time 1 minute accurately, even though they may have different reset intervals. The time that the chip is running software is only about 1%. It is sleeping for the other 99%.

Upon each reset, the software updates its timers and if it is time for another dose, energises the motor M for a defined period, enough to drive the gears and operate the mechanism. This time period has been experimentally determined to give a correct dose even at low temperatures and low battery voltage. Transistor Q1 turns the motor on/off via base resistor R2 and the microcontroller signal ‘MOTOR’. The microcontroller is not able to provide enough current to drive the motor, so a transistor must be used. Capacitors C1 & C2 provide motor suppression.

The software also measures the battery voltage by energising the red LED1 for a very brief period (invisible to the eye) and measuring the voltage drop across the LED. This is the signal labelled ‘SENSE’ on the circuit diagram. Red LEDs have a reasonably stable voltage drop and can be used as a simple voltage reference. Using an existing red LED rather than a separate voltage reference keeps cost to a minimum. By comparing this voltage to the supply voltage of the chip, the battery voltage can be measured indirectly. The unit will only flash its LEDs and operate the motor if the battery voltage is above 2.5v. A comparator within U1 performs this voltage measurement/comparison.

The microswitch state is checked using signal ‘SWITCH’. The motor will only operate if the switch lever is pressed. This actually opens the switch contacts, as the normal operation will be with a canister in position and open contacts gives the lowest power drain from the batteries. When the canister is removed, the contacts are made and the microcontroller software starts timing. If the canister is kept removed for more than 16 seconds and the dose count is at maximum, the dose count is reset to zero. The count will not be reset if the canister is removed and replaced within 16 secs. The count will also not be reset unless the count has reached maximum.

Before the software enters sleep mode, it energises either the red or green LED if the battery voltage is sufficient (>2.5v). If the batteries are low, neither LED is energised. The green LED is energised if the dose count is less than the low warning level. The red LED is energised if the dose count is greater than the low warning level. In addition, if the dose count is at maximum, the watchdog reset interval is decreased fourfold (from 3 secs to 0.75 sec) to give a faster flash rate when the canister is empty.

Both red and green LEDs are energised by taking the signals ‘LOW_CAN_LED’ and ‘POWER_OK’ low respectively. Sinking current is better than sourcing it for giving the brightest light from the LEDs. resistors R3 and R5 control the LED currents. R4 is not fitted. However, it is possible to change the green LED to a bicolour LED and then to fit resistor R4 (and remove resistor R5) to allow two-colour control of the LED. At present, LED2 is a single-colour green LED.

The following list of operations is performed each time the software runs (due to a reset from the watchdog).
1. Initialise all registers of the microcontroller. This will turn off the LEDs.
2. If the unit has been power cycled (off, then on), reset the ‘test dose’ timer. This will ensure a test dose is made each time the unit is switched on.
3. Put the microcontroller into lowest power SLEEP mode. Will wakeup after 3 secs.
4. Update the time variables.
5. If canister is in place, check if it is time for the initial ‘test dose’ (30 secs). If so, signal for a dose to be delivered and update the dose count.
6. If canister is removed, check if it has been removed for more than 16 seconds. If so, reset the dose count (only if the count has reached maximum).
7. Turn the red LED on very briefly and measure the voltage drop using the built-in comparator of the microcontroller. Set the ‘low battery’ flag if battery level too low.
8. Check if it is time for another regular dose (every 2 hours). If so, signal for a dose to be delivered and update the dose count.
9. If a dose has been signalled, turn on motor for N millisecs. Then turn off.
10. Check dose count and ‘low battery’ flag. Turn on red or green LED as appropriate.
11. Calibrate the watchdog period. This time period defines the time that the LEDs are on. As part of the calibration, the microcontroller is allowed to reset, taking the operation back to step 1.

The operation of the device will now be considered. When the unit is switched on, the green LED will flash once every 3 secs if the battery level is good, and the canister is not running low.

After 30 seconds of switch on and with a canister in place, a test dose will be generated to show that the unit is working correctly. Thereafter, doses will be provided every 131 mins (just over 2 hours). This will give 990 doses over 90 days. Doses will only be generated if a canister is in place.

When the dose count exceeds 890, and the batteries are good, the green LED will stop flashing and the red LED will flash once every 3 secs. Once the dose count reaches 990, the flash rate will increase, so that the red LED flashes once every 0.75 sec. This shows that the canister is empty, though the motor will continue to operate every 2 hours, in case there is still product left in the canister.

To reset the dose count, the canister must be removed whilst the unit is still switched on. After 16 seconds without a canister, the red LED will stop flashing at a fast rate and the green LED will start flashing at the slower rate, indicating that the count has been reset. A fresh canister can then be inserted. After inserting, a new test dose is generated after 30 seconds, just like at switch-on.

NOTE: If the unit is switched off and the canister replaced, the unit will have no way of knowing that the change has taken place and therefore when switched back on, the low canister LED will continue to flash. Resetting of the dose count can only be done with the unit switched on.

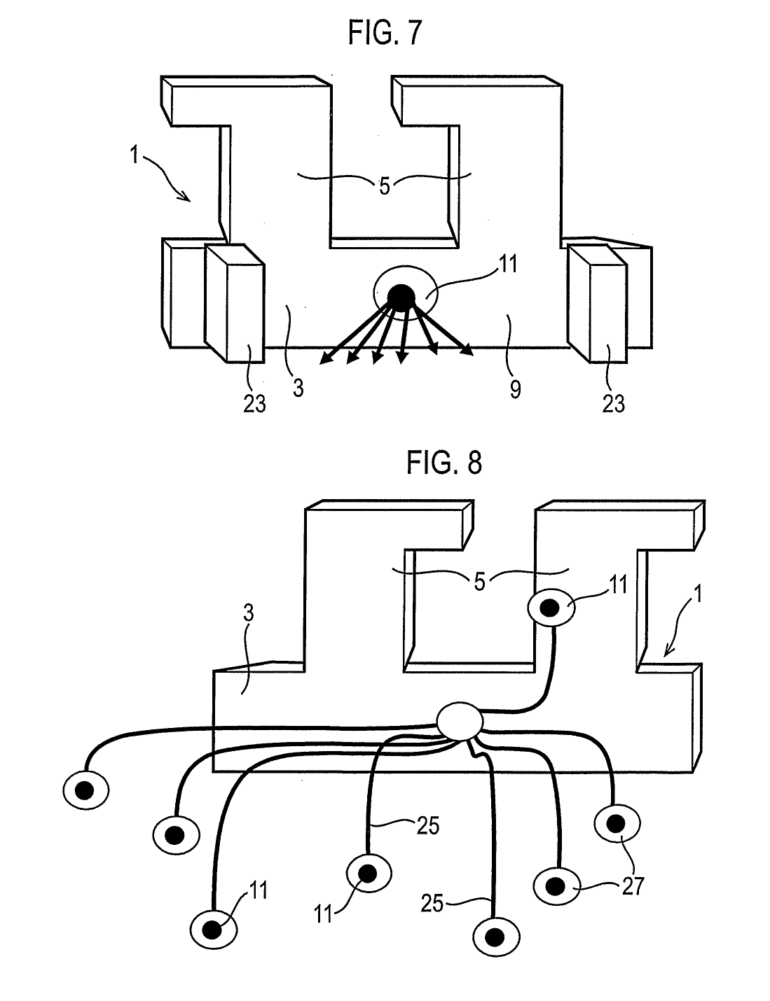

FIG. 7 is a simplified perspective view of one form of the device in which the nozzle 11 is located at the rear of the housing and is arranged to send out the aerosol downwards and at an angle so that none of the contents of the bin can get in the way and block the nozzle and no spray can get into the eyes of a user, In order to ensure a suitable spacing of the nozzle from the rear of the bin against which the device rests, a pair of spacing blocks 23 are located on the rear of the housing 3.

FIG. 8 shows a variant of the device in which a relatively large number of nozzles 11 are used, here eight. The nozzles 11 are located on the ends of flexible pipes 25 so that they can be positioned in the bin in suitable locations. For maintaining the nozzles 11 in their respective chosen positions, the nozzles are provided with suction cups 27. The nozzles 11 can be connected to the suction cups 27 in such a manner that their direction of operation can be varied.

Various different spray materials may be used in the above described device. Suitably, the spray materials will be biodegradable. Any suitable formulation can be used which provides a germicide. Suitably, the formulation will contain a strong smell neutraliser and a soap formulation which will actually cleanse the bin to this end, this formulation would normally be based on soap technology which provides a soap that does not require to be washed off after application. In addition, there would be an insect and vermin repellent and an insect killer might also be provided.

Examples of the materials which could be used are:—
1. Alcohols (such as ethanol or isopropanol)
2. Aldehydes (such as Glutaraldehyde)
3. Halogens (such as Chloramine, Chlorine, Hypochlorites (Sodium hypochlorite), calcium hypochlorite, Hypobromite, Iodine)
4. Oxidizing agents (such as Chlorine dioxide, Sodium chlorite, sodium chlorate, and potassium chlorate, Hydrogen peroxide, Ozone, Acidic Electrolyzed Water, Peracetic acid, Potassium permanganate, Potassium peroxymonosulfate)
5. Phenolics (such as Phenol, O-phenylphenol, Chloroxylenol, Hexachlorophene, Thymol, Quaternary ammonium compounds)
6. Quaternary ammonium compounds (Quats), (such as benzalkonium chloride)
7. Essential oils e.g Agar oil, Ajwain oil, Angelica root oil, Anise oil, Balsam oil, Basil oil, Bergamot oil, Black Pepper, Buchu oil, Cannabis flower essential oil, Caraway oil, Cardamom seed oil, Carrot seed oil Cedarwood oil, Chamomile oil, Cinnamon oil, Cistus, Citronella oil, Clary Sage, Clove leaf oil, Coriander, Costmary oil, Cranberry seed oil, Cumin oil/Black seed oil, Cypress, Davana oil, Dill oil, Eucalyptus oil, Fennel seed oil, Fenugreek oil, Fir, Frankincense oil, Galbanum, Geranium oil, Ginger oil, Goldenrod, Grapefruit oil, Henna oil, Helichrysum, Hyssop, Idaho Tansy, Jasmine oil, Juniper berry oil, Lavender oil, Laurus nobilis, Lavender oil, Ledum, Lemon oil, Lemongrass, Litsea cubeba oil, Marjoram, Melaleuca See Tea tree oil, Melissa oil (Lemon balm), Mentha arvensis oil/Mint oil, Mountain Savory, Mugwort oil, Mustard oil (essential oil), Myrrh oil, Myrtle, Nutmeg, Orange oil, Oregano oil, Orris oil, Palo Santo, Parsley oil, Patchouli oil, Perilla essential oil, Pennyroyal oil, Peppermint oil, Petitgrain, Pine oil, Ravensara, Red Cedar, Roman Chamomile, Rose oil, distilled Rosehip oil, Rosemary oil, Rosewood oil, Sage oil, Sandalwood oil, Sassafras oil, Savory oil, Schisandra oil, Spearmint oil, Spikenard, Spruce, Star anise oil, Tangerine, Tarragon oil, Tea tree oil, Thyme oil, Tsuga, Valerian, Vetiver oil (khus oil), Western red cedar, Wintergreen, Yarrow oil, Ylang-ylang
8. Bleach
9. Biocides
10. Water or other dilatants
11. Aromatic chemicals
12. Emulsifiers
13. Water soluble perfumes
14. Propellants
15. Perfume
16. Ethanol
17. Acetone
18. Butane
19. Propane
20. Sodium Bicarbonate
21. Ammonia
22. Insect repellent
23. Pesticides
24. Insecticide
25. Ovicides
26. Larvicide's
27. Anti-Bacterial agents
28. Anti-Fungal agents
29. Anti-Viral agents
30. Acid
31. Biological surfactants
32. Chloroxylenol

It will be appreciated that additions to or modification of the above described sanitizing device may be made without departing from the scope of the invention. For example, the number of nozzles used on the device can be varied as desired, suitably between 1 and 8 nozzles. The location of the nozzles could also be varied by positioning some on the bottom of the housing and/or on the sides of the housing. It will also be understood that while the described sanitizing device has been described for use with wheelie bins, it could equally well be used with other containers such as dustbins. It will be understood that the bracket 5 is a universal bracket which can be used to support any suitable device, such as a deodorizer which is not mechanically actuated, for deodorising a wheelie bin or other refuse container.

What is claimed as new and desired to be protected by Letters Patent of the United States is:

1. A sanitizing device for a portable refuse container comprising a housing, an atomizing device within said housing and containing sanitizing material, a nozzle attached to said housing for dispersing the sanitizing material outside of said housing, connecting means connecting said atomising device to said nozzle, timing means controlling the operation of said atomising device and attaching means for releasably attaching said housing to the portable refuse container including at least one hook extending upwardly from said housing and adapted to enable said device can be hooked onto a lip of the portable refuse container so as to suspend said housing within the container.

2. A device as claimed in claim 1, wherein said nozzle is positioned on the housing.

3. A device as claimed in claim 1 wherein the nozzle is provided on the end of a flexible pipe connected to the atomising device whereby the nozzle can be arranged to be spaced from the housing.

4. A device as claimed in claim 1, wherein two or more nozzles are provided to give full cover to the contents of the portable refuse container.

5. A device as claimed in claim 4, wherein three nozzles are provided.

6. A device as claimed in claim 4, wherein more than three nozzles are provided and flexible pipes are provided which flexibly attach said nozzles to said device.

7. A device as claimed in claim 1 wherein the timing means comprises an electric timer settable to time predetermined intervals.

8. A device as claimed in claim 7 wherein the timer is adjustable so as to vary the length of the timed intervals.

9. A device as claimed in claim 8, wherein said atomising device is electrically operated and is triggered by said timer.

10. A device as claimed in claim 1 wherein said atomising device comprises an aerosol container containing the sanitizing material.

11. A device as claimed in claim 1, wherein said hook comprises a bracket adapted for attachment of said device to the upper part of a container, said bracket comprising a back plate, attachment wings provided in mirror image on both sides of the back plate cooperating with the slots in the rear of said device and adapted such that, when the bracket is in position, said device can be engaged on said wings and pushed parallel to said bracket to seat said device on said bracket, a flat portion extending from the top of said back plate at right angles to said back plate, a “U” shaped connecting part connecting said flat portion to said back plate, a front plate intended to extend down the outside of the container and means for securing the bracket to a wall of the container.

12. A device as claimed in claim 11, wherein said back plate of said bracket defines an aperture into which a detent on said device passes to secure said device to said bracket

13. A device as claimed in claim 11, wherein said bracket has the dimensions stated in tables 1 and 2 plus or minus ten percent.

14. A device as claimed in claim 11, wherein said bracket has the dimensions stated in tables 1 and 2.

15. A device as claimed in claim 11, wherein said bracket is adapted to be secured to the wall of the container by a screw which is threaded through said front plate and has a tip adapted to engage the wall of the container so as to trap the container wall between said tip and said rear plate

16. A device as claimed in claim 15, wherein a captive nut is provided on said front plate through which said screw is threaded.
